(19) United States
(12) Patent Application Publication
(10) Pub. No.: US 2024/0408303 A1
(43) Pub. Date: Dec. 12, 2024

(54) INFUSION PUMP SYSTEMS AND METHODS WITH INSULIN PRESERVATIVE LOW THRESHOLD ALERT

(71) Applicant: Tandem Diabetes Care, Inc., San Diego, CA (US)

(72) Inventors: Michael Michaud, San Diego, CA (US); Adam Evard, San Diego, CA (US); Brendan Nichols, San Diego, CA (US); Francesca Shadeed, San Diego, CA (US)

(21) Appl. No.: 18/207,094

(22) Filed: Jun. 7, 2023

(51) Int. Cl. A61M 5/172; A61M 5/142; A61M 5/168

(52) U.S. Cl. CPC A61M 5/172; A61M 5/14244; A61M 5/16886; A61M 2205/18; A61M 2205/3334; A61M 2205/3368; A61M 2205/50

(57) ABSTRACT

Disclosed herein are systems and methods for providing an alert to a user when preservative values in insulin formulations are expected to be low. Variables that affect preservative loss in insulin can be tracked by an insulin pump while insulin is being delivered by the pump. If the variables indicate that preservative values are below a threshold, an alert can be provided on the pump instructing the user to replace the insulin and/or implement another corrective action.

### TECHNICAL FIELD

The present disclosure relates to medical pumps for delivering medicament to a patient, and more specifically, to a user-wearable infusion pump that can be worn on the body of a user.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps that are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some pumps utilize an infusion set that includes a cannula inserted into the body of the user in fluid communication with an infusion pump that can be carried by the user such as in the user's pocket, through infusion tubing. Other pumps, often referred to as patch pumps, can be carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site.

Insulin formulations used in such pumps include phenolic preservatives that stabilize the insulin, allow for an extended shelf life and provide sterility to the formulation. However, materials used in some infusion sets and pumps are not ideal for retaining the phenolic preservatives in the insulin formulations. As such, there is an issue with loss of preservatives in insulin formulations during storage and use of insulin pumps.

### SUMMARY

Disclosed herein are systems and methods for providing an alert to a user when preservative values in insulin formulations are expected to be low. Variables that affect preservative loss in insulin can be tracked by an insulin pump while insulin is being delivered by the pump. If the variables indicate that preservative values are below a threshold, an alert can be provided on the pump instructing the user to replace the insulin and/or implement another corrective action.

In an embodiment, a user-wearable infusion pump system can include a pump mechanism configured to deliver insulin to a user and at least one processor configured to initiate delivery of insulin to a user with an insulin pump. The at least one processor can monitor a temperature in the insulin pump during delivery of the insulin and a flow rate of the insulin during delivery of the insulin. The temperature and the flow rate can be compared to one or more thresholds and an alert can be selectively provided to the user to implement a corrective action regarding the insulin if indicated by the comparison of the temperature and the flow rate to the one or more thresholds.

In an embodiment, a method of diabetes therapy includes initiating delivery of insulin to a user with an insulin pump. A temperature in the insulin pump and a flow rate of the insulin can be monitored during delivery of the insulin. The temperature and the flow rate can be compared to one or more thresholds. An alert can be selectively provided to the user to implement a corrective action regarding the insulin if indicated by the comparison of the temperature and the flow rate to the one or more thresholds.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1C depicts an embodiment of a pump system according to the disclosure.

FIGS. 2A-2B depict an embodiment of a pump system according to the disclosure.

FIG. 3 depicts a block diagram of features of a pump system according to the disclosure.

FIG. 4 depicts a flow chart of method steps for providing an alert when preservative values are expected to be low.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

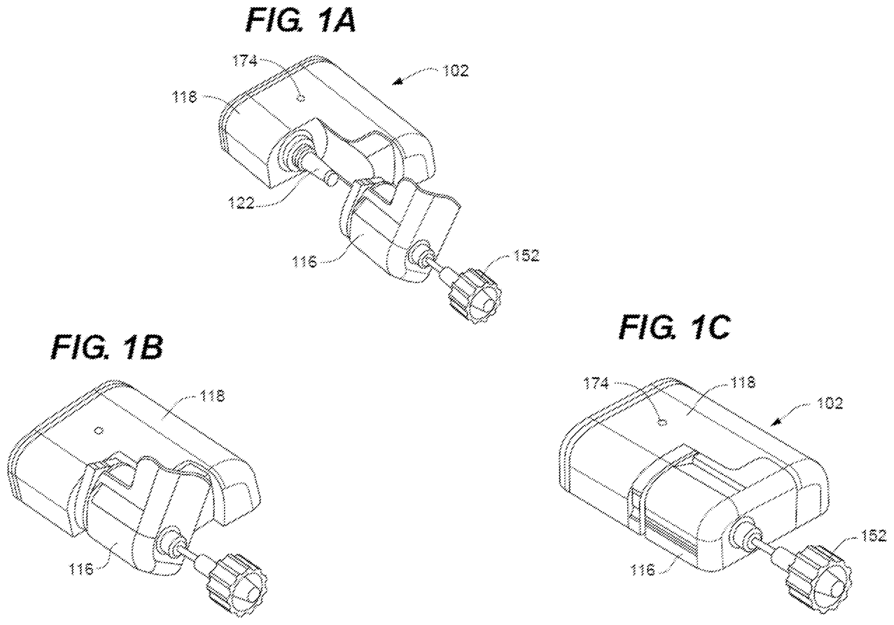

Referring to FIGS. 1A-1C, a pump system 100 including a pump 102 is depicted in accordance with an embodiment of the disclosure. Drive unit 118 of pump 102 includes a drive mechanism 122 that mates with a recess in medicament cartridge 116 of pump 102 to attach the medicament cartridge 116 to the drive unit 118. Further details regarding example embodiments of such delivery mechanisms can be found in U.S. Patent Publication No. 2017/0049957, which is hereby incorporated by reference in its entirety. In some embodiments, cartridge 116 can rotationally attach to drive unit 118 as shown in FIGS. 1B-1C. Further details regarding such rotational attachment can be found in, for example, U.S. Pat. No. 9,993,595, which is hereby incorporated by reference in its entirety. Other pump systems that can be employed with aspects of the disclosure include those described in U.S. Patent Publication No. 2011/0144586, which is hereby incorporated by reference in its entirety.

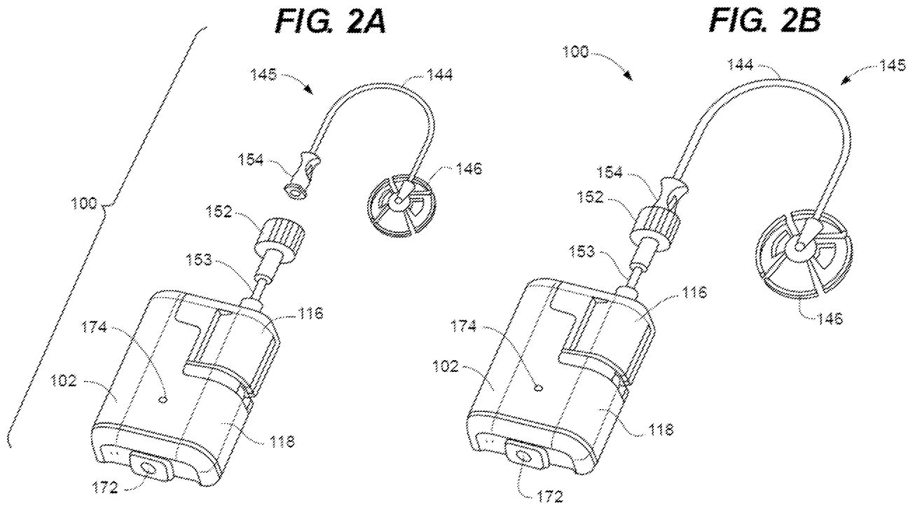

As depicted in the embodiment of FIGS. 2A-2B, pump system 100 can include a pump 102 and an infusion set 145. FIG. 2A depicts this infusion set 145 as not connected to pump while FIG. 2B depicts infusion set 145 connected to pump 102 via connectors 154 and 152 (which extends from the pump 102 via tubing 153). Infusion set 145 can include tubing 144 extending between a connector 154 and a site connector 146. Connector 154 can be configured to couple to pump 102 at connector 152. Site connector 146 can be configured to be attached to an infusion site on a user, while pump 102 can be carried in a separate location, such as the user's pocket or another location on the user's body. Various lengths of tubing 144 can be used in this embodiment to accommodate the user's preference. In other embodiments, pump 102 can be configured to deliver medicament through a cannula extending directly beneath the pump 102. In embodiments, pump can include one or more indicator lights 174 and/or input buttons 172. Further details regarding such pumps can be found in U.S. Pat. Nos. 9,993,595; 10,279,106; and 10,279,107, each of which is hereby incorporated herein by reference in its entirety.

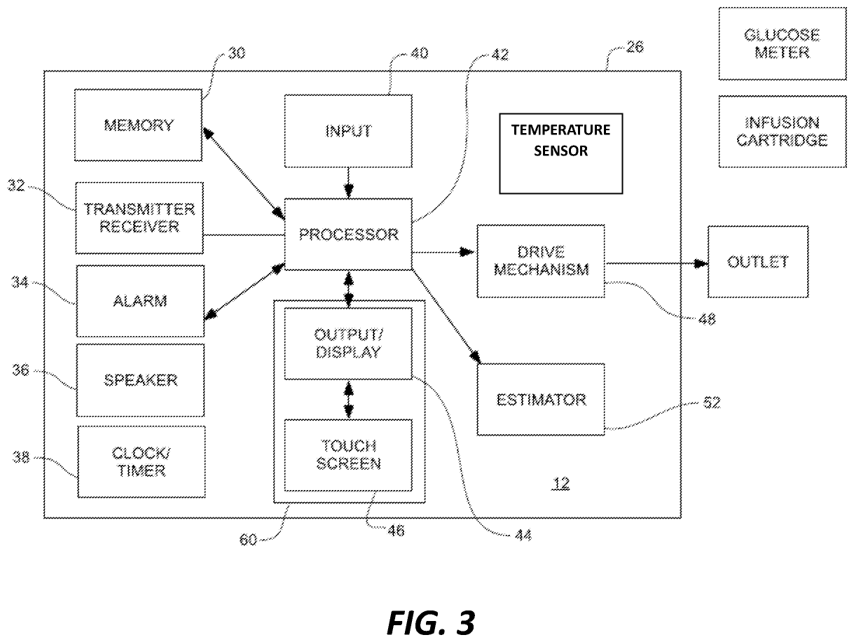

FIG. 3 illustrates a block diagram of some of the features that may be included within a housing 26 of a pump system 12 according to the disclosure. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52, a microphone (not pictured), and a temperature sensor. One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, accelerometer, GPS receiver or the like.

As noted above, materials used in some infusion sets and pumps are not ideal for retaining phenolic preservatives in the insulin formulations that stabilize the insulin, allow for an extended shelf life and provide sterility to the formulation. As such, there is an issue with loss of preservatives in insulin formulations during storage and use of insulin pumps.

Preservative loss can be influenced by a number of factors, including temperature, flow rate of insulin, specifics of the insulin formulation, and the specific materials used in the insulin contacting components of the system (e.g., the insulin reservoir in the pump and the infusion set). Many insulin pumps include one or more temperature sensors and monitor insulin flow rates. Flow rates may be monitored by the processor based on the programmed basal rates delivered, as well as bolus deliveries. Embodiments disclosed herein leverage these existing features of insulin pumps to provide systems and methods for monitoring and/or estimating preservative loss in insulin formulations and for notifying a user when insulin should be replaced due to such preservative loss.

In embodiments, users can be alerted when insulin preservative values are expected to be below a specific threshold by monitoring the pump temperature and delivery rate over time. For example, because preservative loss occurs at higher temperatures and at lower flow rates, if the flow rate of insulin since the cartridge was filled has been below a predetermined threshold of units per day and the average temperature since the cartridge was filled has been above a predetermined threshold temperature, an alert can be provided to the user to, for example, replace the insulin in the cartridge (and/or the cartridge itself in the case of a pre-filled cartridge). In various embodiments, the user can alternatively or additionally be instructed to flush the infusion tubing of insulin and/or replace the infusion tubing. In some embodiments, the system can track different levels of preservative loss of insulin within the cartridge and for insulin that has traveled through the infusion tubing for delivery to the user.

The predetermined thresholds for flow rate and temperature can be variable and dependent on each other. For example, at lower flow rates lower temperatures may trigger an alert. Similarly, when temperatures are higher an alert may be triggered at higher flow rates. In some embodiments, a lookup table can be provided that includes correlated values for the flow rate and the temperature to determine when the combination of the values for the two variables warrants an alert. In various embodiments, the flow rate can be the current flow rate at the time of the temperature reading or an average flow rate over a predetermined period of time prior to the temperature reading. Similarly, the temperature reading can be a temperature reading at a given time or an average temperature over a predetermined amount of time. For example, in some embodiments, an alert can be triggered if the temperature exceeds a specifical level for a minimum amount of time at a given flow rate.

Different thresholds can be used for different types of insulin. Different thresholds can also be used for different infusion set length and type combinations. In some embodiments, the user may enter the type of insulin and/or the infusion set information into the pump. In other embodiments, one or both parameters can be detected by the pump. For example, the system may be able to detect the type of insulin by reading optical indicia or other information on an infusion cartridge containing the insulin. The system may be able to calculate the length and/or internal surfaced area of the infusion set based on a volume of medicament using in priming the pump upon initial attachment of the infusion set.

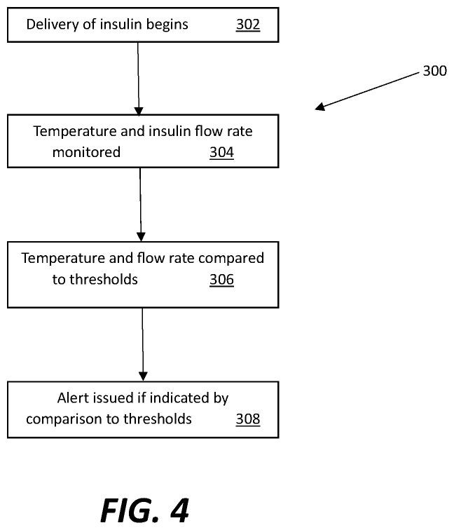

Referring now to FIG. 4, a method 300 for alerting users when preservative values are expected to be low is depicted. At step 302 an insulin pump begins delivering newly filled insulin to a user. This may occur, for example, after the user fills the pump with insulin or when a new insulin cartridge pre-filled with insulin is attached to the pump. At step 304, the system monitors temperature and insulin flow rate during delivery of the insulin. The temperature and flow rate values can be compared to corresponding thresholds at step 306. This can be done, for example, by using a lookup table as described above that includes variable thresholds based on relative values of the temperature and flow rate. In some embodiments, temperature and flow rate can be monitored simultaneously. In other embodiments, one parameter may be monitored and the other parameter reviewed only when the first parameter crosses a predefined threshold.

At step 308, an alert can be issued instructing a user to take a corrective action to replace the insulin in response to preservative loss in the insulin if indicated by the thresholds and the temperature and flow rate values. Such an alert can be provided one or more of visually, audibly, tactilely, etc. The alert can be provided on the pump and/or one or more devices communicatively coupled to a pump, such as, for example, a dedicated remote control device, smartphone, smart watch, smart ring, etc. In some embodiments, a visual alert can provide instructions to the user for how to address the issue of preservative loss, such as replacing the insulin cartridge with a cartridge containing new insulin. Various procedures and mechanisms for providing alerts in infusion pump systems can be found in U.S. Patent Publication Nos. 2020/0306445, 2022/0238201 and 2023/0113545, which are hereby incorporated by reference herein in their entireties.

In embodiments, data and calculations described herein relating to loss of preservatives in insulin can be employed for other uses in the system beyond the above-described alert. For example, preservative degradation correlates with a loss of potency of insulin. This loss of potency can be used with physiological dose calculators such as automated insulin delivery algorithms and bolus calculators for more accurate insulin dosing that takes into account the loss of potency of the insulin in determining the amount of insulin to be delivered to the user.

In embodiments, a user-wearable infusion pump system can include a pump mechanism configured to deliver insulin to a user and at least one processor configured to initiate delivery of insulin to a user with an insulin pump. The at least one processor can monitor a temperature in the insulin pump during delivery of the insulin and a flow rate of the insulin during delivery of the insulin. The temperature and the flow rate can be compared to one or more thresholds and an alert can be selectively provided to the user to implement a corrective action regarding the insulin if indicated by the comparison of the temperature and the flow rate to the one or more thresholds.

In some embodiments, the at least one processor is configured to compare the temperature and the flow rate to one or more thresholds using a lookup table.

In some embodiments, the lookup table compares relative values of temperature and flow rate.

In some embodiments, the at least one processor is configured to provide the alert when relative values of the temperature and the flow rate indicate that preservative values in the insulin are likely to be below a predetermined threshold.

In some embodiments, the at least one processor is configured to compare current values of the temperature and the flow rate.

In some embodiments, the at least one processor is configured to compare an average value of temperature and an average flow rate over a period of time.

In some embodiments, the alert informs the user of preservative loss in the insulin.

In some embodiments, the alert instructs the user to replace the insulin as the corrective action.

In some embodiments, the alert instructs the user to replace the insulin in a user-wearable infusion pump.

In some embodiments, the alert instructs the user to dispose of an insulin cartridge of the user-wearable infusion pump and to replace the insulin cartridge with a new insulin cartridge.

In embodiments, a method of diabetes therapy includes initiating delivery of insulin to a user with an insulin pump. A temperature in the insulin pump and a flow rate of the insulin can be monitored during delivery of the insulin. The temperature and the flow rate can be compared to one or more thresholds. An alert can be selectively provided to the user to implement a corrective action regarding the insulin if indicated by the comparison of the temperature and the flow rate to the one or more thresholds.

In some embodiments, comparing the temperature and the flow rate to one or more thresholds includes using a lookup table.

In some embodiments, the lookup table compares relative values of temperature and flow rate.

In some embodiments, the alert is selectively provided when relative values of the temperature and the flow rate indicate that preservative values in the insulin are likely to be below a predetermined threshold.

In some embodiments, comparing temperature and flow rate includes comparing current values of the temperature and the flow rate.

In some embodiments, comparing temperature and flow rate includes comparing an average value of temperature and an average flow rate over a period of time.

In some embodiments, the alert informs the user of preservative loss in the insulin.

In some embodiments, the alert instructs the user to replace the insulin as the corrective action.

In some embodiments, the alert instructs the user to replace the insulin in a user-wearable infusion pump.

In some embodiments, the alert instructs the user to dispose of an insulin cartridge of the user-wearable infusion pump and to replace the insulin cartridge with a new insulin cartridge.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. A user-wearable infusion pump system, comprising:
a pump mechanism configured to deliver insulin to a user; and
at least one processor configured to:
initiate delivery of insulin to a user with an insulin pump;
monitor a temperature in the insulin pump during delivery of the insulin;
monitor a flow rate of the insulin during delivery of the insulin;
compare the temperature and the flow rate to one or more thresholds; and
selectively provide an alert to the user to implement a corrective action regarding the insulin if indicated by the comparison of the temperature and the flow rate to the one or more thresholds.

2. The system of claim 1, wherein the at least one processor is configured to compare the temperature and the flow rate to one or more thresholds using a lookup table.

3. The system of claim 2, wherein the lookup table compares relative values of temperature and flow rate.

4. The system of claim 1, wherein the at least one processor is configured to provide the alert when relative values of the temperature and the flow rate indicate that preservative values in the insulin are likely to be below a predetermined threshold.

5. The system of claim 1, wherein the at least one processor is configured to compare current values of the temperature and the flow rate.

6. The system of claim 1, wherein the at least one processor is configured to compare an average value of temperature and an average flow rate over a period of time.

7. The system of claim 1, wherein the alert informs the user of preservative loss in the insulin.

8. The system of claim 1, wherein the alert instructs the user to replace the insulin as the corrective action.

9. The system of claim 8, wherein the alert instructs the user to replace the insulin in a user-wearable infusion pump.

10. The system of claim 9, wherein the alert instructs the user to dispose of an insulin cartridge of the user-wearable infusion pump and to replace the insulin cartridge with a new insulin cartridge.

11. A method of providing diabetes therapy, comprising:
initiating delivery of insulin to a user with an insulin pump;
monitoring a temperature in the insulin pump during delivery of the insulin;
monitoring a flow rate of the insulin during delivery of the insulin;
comparing the temperature and the flow rate to one or more thresholds; and
selectively providing an alert to the user to implement a corrective action regarding the insulin if indicated by the comparison of the temperature and the flow rate to the one or more thresholds.

12. The method of claim 1, wherein comparing the temperature and the flow rate to one or more thresholds includes using a lookup table.

13. The method of claim 12, wherein the lookup table compares relative values of temperature and flow rate.

14. The method of claim 11, further comprising selectively providing the alert when relative values of the temperature and the flow rate indicate that preservative values in the insulin are likely to be below a predetermined threshold.

15. The method of claim 11, wherein comparing temperature and flow rate includes comparing current values of the temperature and the flow rate.

16. The method of claim 11, wherein comparing temperature and flow rate includes comparing an average value of temperature and an average flow rate over a period of time.

17. The method of claim 11, wherein the alert informs the user of preservative loss in the insulin.

18. The method of claim 11, wherein the alert instructs the user to replace the insulin as the corrective action.

19. The system of claim 18, wherein the alert instructs the user to replace the insulin in a user-wearable infusion pump.

20. The system of claim 19, wherein the alert instructs the user to dispose of an insulin cartridge of the user-wearable infusion pump and to replace the insulin cartridge with a new insulin cartridge.
